(19) United States
(12) Patent Application Publication
(10) Pub. No.: US 2022/0243021 A1
(43) Pub. Date: Aug. 4, 2022

(54) POLYMERIC CELL CULTURING SURFACE HAVING HIGH CELL ADHESION

(71) Applicant: SIO2 MEDICAL PRODUCTS, INC., Auburn, AL (US)

(72) Inventors: Ahmad Taha, Auburn, AL (US); Brian Maurer, Auburn, AL (US); Matthew Wills, Auburn, AL (US)

(21) Appl. No.: 17/268,884

(22) PCT Filed: Dec. 7, 2018
(86) PCT No.: PCT/US2018/064617

§ 371 Date: Feb. 16, 2021

Related U.S. Application Data
(60) Provisional application No. 62/718,284, filed on Aug. 13, 2018.

(51) Int. Cl. C08J 7/12; C12M 1/24; C12M 1/00; C12M 1/12; C12M 3/04

(52) U.S. Cl. CPC C08J 7/123; C12M 23/08; C12M 23/20; C08J 2325/06; C12M 25/06; C12M 27/12; C08J 2300/22; C12M 25/02

(57) ABSTRACT

A polymeric substrate has a plasma treated contact surface which is roughened, hydrophilic and has a higher oxygen atomic content than the interior portion. The treated contact surface has an enhanced cell adhesion, cell growth and cell recovery rate. During the treatment, the contact surface is contacted with a process gas introduced through a gas inlet near the treated surface and radio frequency electrical power is introduced in the process gas, forming a treated contact surface that has improved cell recovery compared to an untreated contact surface. The process gas optionally can be nitrogen gas, oxygen gas, or a gas that contains nitrogen atoms, oxygen atoms, or a combination of nitrogen and oxygen atoms. The process optionally improves cell recovery of a chicken embryo cell culture from the treated contact surface.

### CROSS-REFERENCE TO RELATED APPLICATIONS

This application is a U.S. National Phase of International Application No. PCT/US2018/064617 filed Dec. 7, 2018, which claims priority to U.S. Provisional Patent Application No. 62/718,284 filed Aug. 13, 2018 and which is continuation-in part of International Application No. PCT/US2018/056722, filed Oct. 19, 2018 which are incorporated herein by reference in their entirety.

### FIELD OF INVENTION

The technology relates generally to a surface, or surface treatment of a plastic substrate (sometimes referred to in this disclosure as a contact surface), that is rough and optionally hydrophilic, or to methods for making the surface roughened and optionally hydrophilic and enhancing cell adhesion to the surface. More particularly, the technology relates to a plastic substrate, e.g. a medical device or item of laboratory ware, with a treated contact surface used as a substrate for cell culture and cell growth due to its enhanced cell adhesion. Such medical devices include but are not limited to cell culture vessels and roller bottles.

This invention also relates to plasma treated cell growth and cell culture vessels and plastic lab ware. This invention further relates to a rough and optionally hydrophilic surface provided, for example, by plasma treatment. This invention further relates to generation of a roughened and optionally hydrophilic surface with enhanced cell adhesion and thereby an improved cell culture and cell growth.

### BACKGROUND

Although some cells grow in suspension (e.g. 3D sphere culture suspension), such as hematopoietic cell lines and transformed cells, most other cells grow in favor of high surface binding (e.g. monolayer growth); that is, they must be adhered to a substrate to proliferate.

Surface hydrophilicity is favored for cell adhesion. Traditionally glassware presents a hydrophilic surface and therefore was used and continues to be used for cell culture and cell growth. However, glassware is readily breakable, very expensive, prone to particulate problems, yields heavy metal extractables due to the composition of the glass, and can cause adverse effects on cell growth and/or aggregation of proteins and other biologics.

Some of these problems can be addressed by substituting injection molded plastic ware for glassware. Roller bottles are used as cell culture vessels in a wide variety of applications. Roller bottles are often made from polystyrene (PS) or polyethylene terephthalate (PET). These materials present superior optical clarity, high stability, reduced breakage and many other advantages. Plastic ware addresses some of the problems with glassware, but plastic ware creates certain problems as well.

As stated above, high cell adhesion is considered to enhance cell growth. Therefore, in cell growth vessels, it is desirable to enhance cell adhesion to the plastic ware used with biological substances. High contact surface area and hydrophilicity are beneficial to cell adhesion. Surfaces of common laboratory ware components made of polymeric plastic are smooth and hydrophobic and usually don't have good cell adhesion. These issues limit the use of plastic ware for cell culture vessels and roller bottles.

It is thus a desire to provide surfaces for plastic cell culture/cell growth vessels with high contact surface area and hydrophilicity and thereby improved cell adhesion.

To expand the contacting surface, some roller bottles are designed with circumferential, axial, or other ribs on the body, which can increase the growth surface. Roughening is an efficient method to increase contact surface area. When a contact surface is roughened, more contact surface area becomes available to cell adhesion.

To generate a hydrophilic surface that is beneficial for cell growth, some hydrophilic coatings, including polyethylene glycol (PEG) and zwitterion polymeric coatings are being used which provide good cell adhesion. Many of these polymeric coatings have potential to move (dissolve, disperse) into the fluid payload, causing interference with cell growth or testing, limiting their utility.

### SUMMARY OF THE INVENTION

There is therefore a need for plastic laboratory ware such as a cell culture vessel or a roller bottle, presenting a contact surface with high surface area and being hydrophilic, that will enhance the cell adhesion to the surface of the plastic Likewise, there is a need for such plastic laboratory ware such as a cell culture vessel and a roller bottle having a stable contact surface that will prevent material movement of the treated contact surface thereby preventing undesired particulate interference and exposure of the plastic surface.

The current invention addresses the above issues with providing a plasma treated plastic substrate with a contact surface which is roughened, having a high oxygen content and being hydrophilic and thereby having an enhanced cell adhesion and cell recovery rate.

An aspect of the invention is a polymeric substrate consisting essentially of a contact surface and an interior portion, in which the contact surface has a roughness quantified by at least one of the four parameters below:
A surface area difference A, greater than 0.055%, optionally from 0.06% to 2%, optionally from 0.1% to 1.5%, optionally from 0.5% to 1.2%, optionally from 0.9% to 1.1%;
a root mean square surface slope Sdq, greater than 1.9°, optionally from 2° to 20°, optionally from 4° to 15°, optionally from 6° to 12°, optionally from 7° to 10°, optionally from 7° to 9°;
a density of summits, Sds, greater than 44.4/μm², optionally from 45/μm² to 200/μm², optionally from 50/μm² to 180/μm², optionally from 60/μm² to 170/μm², optionally from 70/μm² to 160/μm², optionally from 80/μm² to 160/μm², optionally from 90/μm² to 150/μm², optionally from 100/μm² to 150/μm², optionally from 110/μm² to 150/μm², optionally from 120/μm² to 150/μm²; or
A mean summit curvature, Ssc, greater than 3.62/μm, optionally from 4/μm to 50/μm, optionally from 6/μm to 45/μm, optionally from 8/μm to 40/μm, optionally from 10/μm to 35/μm, optionally from 12/μm to 30/μm, optionally from 14/μm to 30/μm, optionally from 16/μm to 30/μm, optionally from 18/μm to 30/μm, optionally from 20x/μm to 25/μm;
   wherein the parameters A, Sdq, Sds, and Ssc are determined by measurement of a 5 μm×5 μm image area on the contact surface.

### BRIEF DESCRIPTION OF DRAWING FIGURES

In the drawings,

FIG. 1 is a schematic view of plasma treatment apparatus useful for plasma treatment of contact surfaces.

FIG. 2 is a view similar to FIG. 1 showing plasma treatment apparatus for treating three vessels simultaneously.

FIG. 3 is a schematic sectional view of the apparatus of FIG. 1, showing internal details of the apparatus and an additional feature for equalizing pressure inside and outside of a vessel being treated.

FIG. 4 shows a perspective view of a CELLBIND® roller bottle. CELLBIND® is a registered trademark of Corning Incorporated of Corning, N.Y.

FIG. 5 shows a photographic view similar to FIG. 4 of a commercial roller bottle having circumferential ribs inside and outside its wall, expanding the surface area for cell attachment.

FIG. 6 shows the CELLTREAT™ roller bottle of FIG. 5 as referred to in Example 2 of this specification, identifying relevant parts of the bottle.

FIGS. 7A and 7B show two examples of aseptic caps which can be used to close the vessel of the current invention. FIG. 7A shows a Corning® aseptic transfer cap and FIG. 7B shows a Sartorius MYCAP® closure.

The following reference characters are used in the drawings:

| | |
|---|---|
| 101 | polymeric substrate |
| 102 | contact surface |
| 103 | interior portion (adjacent to the contact surface) |
| 104 | process gas |
| 105 | vessel |
| 106 | wall (of 105) |
| 107 | inner surface (of 106) |
| 108 | lumen (of 105) |
| 109 | outer surface (of 106) |
| 110 | ribs |
| 111 | gas inlet conduit |
| 112 | outlet (of 111) |
| 113 | external applicator |
| 114 | internal applicator |
| 115 | ceramic chamber |
| 116 | aluminum bottom |
| 117 | aluminum lid |
| 118 | pumping port |
| 119 | vacuum conduit |
| 120 | vacuum pump |
| 121 | Valve |
| 122 | processing area |
| 123 | gas system |
| 124 | mass flow controller |
| 125 | matching network |
| 126 | power supply |
| 127 | coaxial cable |
| 128 | vacuum bypass line |
| 129 | valve (of 128) |

Like reference characters indicate corresponding parts.

### DETAILED DESCRIPTION

The term “contact surface” indicates a surface that is in a position to come in contact with a sample or other material, and has surface properties determining its interaction with the sample or other material with which it comes into contact. Some examples of contact surfaces are part or all of a surface of a vessel (for example, bounding a vessel lumen) or an exterior surface of a vessel, sheet, block, or other object. Optionally, the contact surface is made of the same material as the interior portion before the contact surface is treated with plasma.

The term “interior portion” indicates a portion of a bulk article or coating that is not a contact surface, but instead forms part of the interior of the bulk article or coating.

“Plasma,” as referenced in any embodiment, has its conventional meaning in physics of one of the four fundamental states of matter, characterized by extensive ionization of its constituent particles, a generally gaseous form, and incandescence (i.e. it produces a glow discharge, meaning that it emits light). For “direct” plasma (as opposed to remote plasma), substantial amounts of ions and electrons of plasma are in direct contact with the treated article surface.

A treated contact surface is defined for all embodiments as a contact surface that has been plasma treated as described in this specification, and that exhibits enhanced cell growth as a result of such treatment.

The term “vessel” as used throughout this specification may be any type of article that is adapted to contain or convey a liquid, a gas, a solid, or any two or more of these. One example of a vessel is an article with at least one opening (e.g., one, two or more, depending on the application) and a wall including an interior contact surface.

The present disclosure is directed to a substrate made of polymer and having a contact surface and an interior portion. The contact surface has a roughness quantified by at least one of the four parameters below:
A surface area difference A, greater than 0.055%, optionally from 0.06% to 2%, optionally from 0.1% to 1.5%, optionally from 0.5% to 1.2%, optionally from 0.9% to 1.1%;
a root mean square surface slope, Sdq, greater than 1.9°, optionally from 2° to 20°, optionally from 4° to 15°, optionally from 6° to 12°, optionally from 7° to 10°, optionally from 7° to 9°;
a density of summits, Sds, greater than 44.4/μm², optionally from 45/μm² to 200/μm², optionally from 50/μm² to 180/μm², optionally from 60/μm² to 170/μm², optionally from 70/μm² to 160/μm², optionally from 80/μm² to 160/μm², optionally from 90/μm² to 150/μm², optionally from 100/μm² to 150/μm², optionally from 110/μm² to 150/μm², optionally from 120/μm² to 150/μm²; or
A mean summit curvature, Ssc, greater than 3.62/μm, optionally from 4/μm to 50/μm, optionally from 6/μm to 45/μm, optionally from 8/μm to 40/μm, optionally from 10/μm to 35/μm, optionally from 12/μm to 30/μm, optionally from 14/μm to 30/μm, optionally from 16/μm to 30/μm, optionally from 18/μm to 30/μm, optionally from 20x/μm to 25/μm;
   wherein the parameters A, Sdq, Sds, and Ssc are determined by measurement of a 5 μm×5 μm image area on the contact surface.

The contact surface optionally has a roughness quantified by any two of the above four values, alternatively any three of the above four values, alternatively all of the above four values.

The polymeric substrate includes, in addition to the contact surface, an interior portion adjacent to the contact. The contact surface is formed after treatment of the initial surface. The initial surface is the surface prior to the treatment.

Optionally, after the treatment, the XPS atomic composition of the interior portion comprises less oxygen and more carbon than the XPS atomic composition of the contact surface. In other words, after the treatment, the contact surface comprises more oxygen than the interior portion. Optionally, the XPS atomic composition of the contact surface comprises from 0.1 to 30 atomic %, optionally from 2 to 30 atomic %, optionally from 5 to 20 atomic %, optionally from 10 to 20 atomic %, optionally from 13 to 16 atomic % more oxygen than the XPS atomic composition of the interior portion. Optionally oxygen atoms are grafted into the contact surface. Optionally the XPS atomic composition of the contact surface comprises from 0.1 to 20 atomic %, optionally from 5 to 15 atomic %, optionally from 9 to 12 atomic %, carbon atoms to which oxygen is grafted.

Optionally, the treatment is a plasma treatment using RF as the power. Optionally at least during a portion of the time of the treatment, the inlet is inserted into the lumen of the substrate.

Optionally, the contact surface has a thickness of 0 to 20 nm, optionally 0.1 to 10 nm, optionally 0.2 to 1 nm, optionally 0.2 to 0.7 nm, optionally about 0.6 nm; and in which the contact surface is hydrophilic and has higher cell adhesion than untreated otherwise identical surface or biological coating coated surface or a microwave plasma treated surface; and the interior portion comprises essentially carbon and hydrogen atoms.

Optionally, the contact surface has a thickness of less than 0.6 nm and the XPS atomic composition of the interior portion at a depth of 0.6 nm comprises from 1% to 10% oxygen.

Optionally, after the treatment, the contact surface is hydrophilic and has a lower contact angle than untreated. Optionally the contact surface has a contact angle of from 38° to 62°, optionally from 50° to 70°, optionally from 55° to 65°, optionally from 60° to 64°, optionally from 30° to 50°, optionally from 30 to 40°, optionally from 35° to 45°, optionally from 37° to 41°.

Optionally, the substrate comprises a vessel having a wall having an inner surface enclosing a lumen, an outer surface, and an interior portion between and spaced from at least the inner surface and the outer surface. Optionally, the vessel can be, for example, a roller bottle, a ribbed roller bottle, a plate, a dish, a flask, a bottle, or a tube. Optionally the substrate is used for cell growth.

Optionally, the substrate is made of thermoplastic material comprising a hydrocarbon polymer, for example an olefin polymer, polypropylene (PP), polyethylene (PE), cyclic olefin copolymer (COC), cyclic olefin polymer (COP), polymethylpentene, polystyrene, hydrogenated polystyrene, polycyclohexylethylene (PCHE), or combinations of two or more of these, or a heteroatom-substituted hydrocarbon polymer, for example a polyester, polyethylene terephthalate (PET), polyethylene naphthalate, polybutylene terephthalate (PBT), polyvinylidene chloride (PVdC), polyvinyl chloride (PVC), polycarbonate, polylactic acid, epoxy resin, nylon, polyurethane polyacrylonitrile, polyacrylonitrile (PAN), an ionomeric resin, or any combination, composite, blend, or laminate of any two or more of the above materials; optionally polystyrene.

Optionally the substrate is treated with plasma generated by RF. Optionally the contact surface is treated with a process comprising contacting the contact surface with a process gas; and introducing radio frequency (RF) electrical power in the process gas adjacent to the contact surface to generate plasma adjacent to the contact surface, thereby forming a treated polymeric substrate having a treated contact surface. Optionally, the treatment is effective to improve cell recovery from the treated contact surface, relative to initial contact surface. Optionally the cell is a chicken embryo cell culture. The process optionally improves cell recovery of a chicken embryo cell culture from the treated contact surface, relative to the initial contact surface, optionally resulting in cell recovery from the treated contact surface of at least 132%, optionally from 132% to 300%, optionally from 140% to 250%, optionally from 140% to 230%. The cells are grown in Medium DMEM containing calf serum and later were harvested using Trypsin with 0.18 mM EDTA and counted according to the specification.

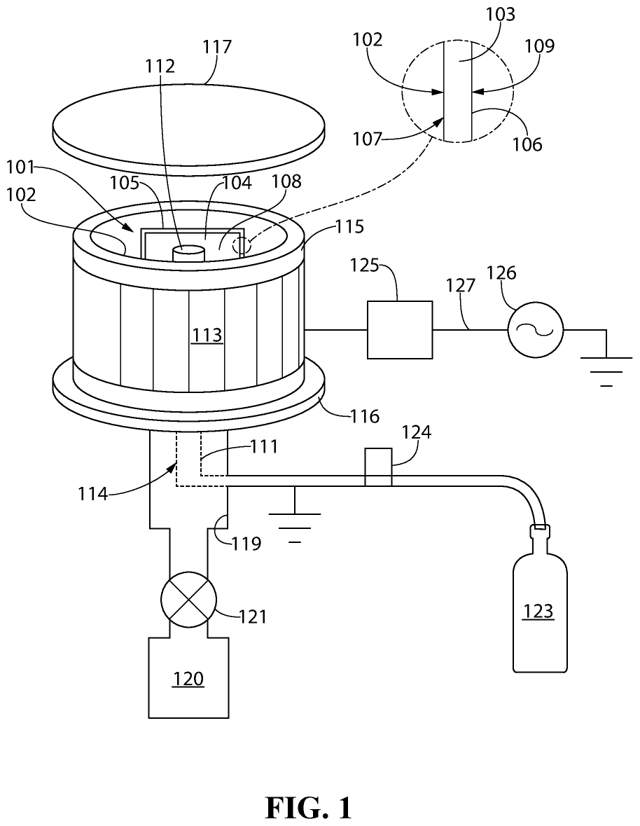

Optionally the plasma is a direct plasma (as opposed to remote plasma). For direct plasma, substantial amounts of ions and electrons of plasma are in direct contact with the treated article surface. Optionally the process gas is essentially free of water. Optionally, during the plasma treatment, the surface is contacted with a process gas by conveying the process gas through a gas inlet conduit having an outlet adjacent to the initial contact surface. Optionally, the gas is introduced into the vessel through a gas inlet inserted into the vessel (as illustrated in FIGS. 1-3).

The process gas optionally can be nitrogen gas, oxygen gas, or a heterogeneous gas that contains nitrogen atoms, oxygen atoms, or a combination of nitrogen and oxygen atoms, as well as other kinds of atoms, for example noble gases. Non-limiting examples of suitable process gas include oxygen gas, nitrogen gas, nitrous oxide gas, or a combination of any two or more of these.

Optionally, the radio frequency electrical power is introduced in the process gas adjacent to the initial contact surface to generate plasma adjacent to the initial contact surface. As a result, a treated polymeric substrate is formed having a treated contact surface.

Optionally, after the treatment, the contact surface has a higher Surface Area Difference A1 value, relative to the Surface Area Difference A2 value of the initial untreated surface. Optionally A1>2×A2, optionally A1>5×A2, optionally A1>10×A2, optionally A1>15×A2, optionally A1>20×A2, optionally A1>25×A2, optionally A1>30×A2, optionally A1>2×A2, optionally A1>35A2, optionally A1>40×A2, optionally A1>50×A2, optionally A1>60×A2, optionally A1>70×A2, optionally A1>80×A2, optionally A1>90×A2, optionally A1>100×A2, optionally A1>110×A2.

Corning® CellBIND® roller bottler (described in U.S. Pat. No. 6,617,152) generated a different contact surface from the current invention. Particularly the contact surface of the current invention has high density of small grain-like structures which are absent from the treated contact surface of the Corning® CellBIND® roller bottler. This difference can be quantified by at least one of the following roughness parameters and as described in Example 5.

Optionally, after the treatment, the contact surface has a higher Surface Area Difference A1 value, relative to the Surface Area Difference A2 value of the interior surface of the 2L Corning® CellBIND® roller bottler. Optionally A1 is greater than 0.055%. Optionally A1>2×A2, optionally A1>5×A2, optionally A1>10×A2, optionally A1>15×A2, optionally A1>20×A2, optionally A1>25×A2, optionally A1>30×A2.

Optionally, after the treatment, the contact surface affords the recovery of a chicken embryo cell culture grown in contact with the treated contact surface and harvested, relative to the initial contact surface, is at least 132%, optionally from 132% to 300%, optionally from 140% to 250%, optionally from 140% to 230%.

Optionally, after the treatment, the contact surface affords the viability of a chicken embryo cell culture grown in contact with the treated contact surface and harvested, relative to the initial contact surface, is at least 88%, optionally from 88% to 99%, optionally from 88% to 97%, optionally from 94% to 96%.

It is a surprising discovery that the surface roughness, higher oxygen atomic composition and hydrophilicity of the contact surface, all generated by the plasma treatment according to the current invention, combined to effectively increase the cell adhesion, cell growth and cell recovery. The surface roughness is characterized by high density of small, grain-like structures, quantified by at least one of the roughness parameters of Surface Area Difference (A), Root Mean Square Surface Slope (Sdq), Density of Summits (Sds) and Mean Summit Curvature (Ssc).

Atomic Force Microscopy (AFM) is used to evaluate the roughness of the surface. The roughness can be quantified by parameters such as Surface Area Difference, Root Mean Square Surface Slope (S_{dq}), or Density of Summits (S_{ds}).

For a same substrate, these parameters may vary depending on the size of the image taken. In order to compare the roughness of two surfaces, the image being taken from each sample should be the same size.

The substrate of the current invention, after treatment, has a contact surface with a higher Surface area difference value (A1), a higher Root Mean Square Surface Slope (Sdq), a higher Density of Summits (Sds) or a Mean Summit Curvature (Ssc). The inventors have also observed, in at least some instances, that the treated contact surface has a higher density of small, grain-like structures than the untreated contact surface. While not intending to be bound by the accuracy of this theory, the inventors theorize that a high density of small, grain-like structures on the treated contact surface according to the current invention can increase the contact surface area more efficiently than the lower density of wider spaced bump structures on the contact surface of the Corning® CellBIND® roller bottler.

### A1

“Imaged area” is the area of a plan view of the area studied, assuming complete flatness.

“Surface area” is the 3-dimensional surface area of the imaged area, taking into account deviations from flatness that increase the surface area. It is calculated by taking the sum of the areas of the triangles formed by 3 adjacent data points throughout the image.

“Surface Area Difference,” A, is the amount that the Surface area in excess of the imaged area. It is expressed as a percentage and is calculated according to the formula:

Surface area difference=100[(Surface area/S₁*S₁)−1]

where S1 is the length (and width) of the scanned area minus any areas excluded by stopbands.

The present disclosure is also directed to a substrate made of polymer having a contact surface and an interior portion. The contact surface has a roughness quantified by at least one of the four parameters below:
A Surface Area Difference A, optionally from 1% to 20%, optionally from 5% to 15%, optionally from 10% to 13%, optionally from 11% to 13%;
a root mean square surface slope (S_{dq}) value, optionally from 10° to 30°, optionally from 15° to 30°, optionally from 20° to 30°, optionally from 25° to 30°, optionally from 26° to 28°;
a density of summits (S_{ds}) value, optionally from 1000/μm² to 3000/μm², optionally from 1500/μm² to 2500/μm², optionally from 1500/μm² to 2000/μm², optionally from 1700/μm² to 1900/μm²; or
A mean summit curvature (Ssc) value, optionally from 500/μm to 800/μm, optionally from 600/μm to 800/μm, optionally from 700/μm to 800/μm;
   wherein a 2μm×0.5 μm area is imaged on the surface.

The contact surface optionally has a roughness quantified by any two of the above four values, alternatively any three of the above four values, alternatively all of the above four values.

Optionally, the Surface Area Difference A1 of the contact surface is larger than the Surface Area Difference A2 of an untreated otherwise identical contact surface. optionally A1>2×A2, optionally A1>5×A2, optionally A1>10×A2, optionally A1>15×A2, optionally A1>20×A2, optionally A1>25×A2, optionally A1>30×A2, optionally A1>2×A2, optionally A1>35A2, optionally A1>40×A2, optionally A1>50×A2, optionally A1>60×A2, optionally A1>70×A2, optionally A1>80×A2, optionally A1>90×A2, optionally A1>100×A2, optionally A1>110×A2.

### Sdq

Root Mean Square Surface Slope (Sdq) is a measure of the slopes that make up the surface texture, evaluated over all directions. It includes amplitude and spacing components.

$$= \sqrt{[ 1 / {\int }_{0} Ly {\int }_{0} Lx \{ {( \partial / \partial ( ) )}^{2} + {( \partial / \partial ( ) )}^{2} \} ]}$$

Lower Sdq values may indicate wider spaced textural components.

Optionally in any embodiment, the Sdq1 of the contact surface is larger than the Sdq2 of an untreated otherwise identical contact surface, optionally Sdq1>2×Sdq2, optionally Sdq1>3×Sdq2 (i.e., Sdq1 is more than three times as great as Sdq2), optionally Sdq1>4×Sdq2, optionally Sdq1>5×Sdq2, optionally Sdq1>6×Sdq2, optionally Sdq1>7×Sdq2, optionally Sdq1>8×Sdq2, optionally Sdq1>9×Sdq2, optionally Sdq1>10×Sdq2, optionally Sdq1>11×Sdq2.

Density of Summits (Sds) is the number of summits per unit area. Summits are derived from peaks. A peak is defined as any point, in a rectilinear array of contiguous points that extends above all 8 of its nearest neighbors. Summits are constrained to be separated by at least 1% of the minimum “X” or “Y” dimension comprising the 3D measurement area. Additionally, summits are only found above a threshold that is 5% of maximum height above the mean plane.

Optionally in any embodiment, the Sds1 of the contact surface is larger than the Sds2 of an untreated otherwise identical contact surface, optionally Sds1>2×Sds2, optionally Sds1>3×Sds2, optionally Sds1>4×Sds2, optionally Sds1>5×Sds2, optionally Sds1>6×Sds2, optionally Sds1>7×Sds2, optionally Sds1>8×Sds2, optionally Sds1>9×Sds2, optionally Sds1>10×Sds2, optionally Sds1>11×Sds2, optionally Sds1>12×Sds2.

Mean Summit Curvature (Ssc) is the mean summit curvature for the various peak structures. Ssc is evaluated for each summit and then averaged over the area:

Ssc=1/N∫∫[{(∂2z(x,y))/∂x2}+{(∂2z(x,y))/∂y2}dxdy]

Optionally in any embodiment, the Ssc1 of the contact surface is larger than the Ssc2 of an untreated otherwise identical contact surface, optionally Ssc1>2×Ssc2, optionally Ssc1>3×Ssc2, optionally Ssc1>4×Ssc2, optionally Ssc1>5×Ssc2, optionally Ssc1>6×Ssc2, optionally Ssc1>7×Ssc2, optionally Ssc1>8×Ssc2, optionally Ssc1>9×Ssc2, optionally Ssc1>10×Ssc2, optionally Ssc1>11×Ssc2, optionally Ssc1>12×Ssc2.

### Rq and Ra

RMS (Rq) is the standard deviation of the Z values (or RMS roughness) in the image. It is calculated according to the formula:

Rq=√{Σ(Zi−Zavg)2/N}

where Zavg is the average Z value within the image; Zi is the current value of Z; and N is the number of points in the image. This value is not corrected for tilt in the plane of the image; therefore, planefitting or flattening the data will change this value.

Optionally in any embodiment, the Rq1 of the contact surface is larger than the Rq2 of an untreated otherwise identical contact surface.

Mean roughness (Ra) is the mean value of the surface relative to the Center Plane and is calculated using the formula:

Ra=[1/(LxLy)]∫0Ly∫0Lx{f(x,y)}dxdy

where f(x,y) is the surface relative to the Center Plane, and Lx and Ly are the dimensions of the surface.

Optionally in any embodiment, the Ra1 of the contact surface is larger than the Ra2 of an untreated otherwise identical contact surface.

The substrate of the current invention, after treatment, has a contact surface with high density of small, grain-like structures which results in higher Root Mean Square Surface Slope (S_{dq}), higher Density of Summits (Sds) and Mean Summit Curvature (Ssc).

### Oxygen Species

Optionally in any embodiment, the XPS atomic composition of the interior portion 103 of the treated polymeric substrate 101 comprises less oxygen and more carbon than the treated contact surface 102.

Optionally in any embodiment, the x-ray photoelectron spectroscopy XPS atomic composition of the treated contact surface 102 is:
from 10% to 25% oxygen, from 0 to 5% nitrogen, and from 70% to 90% carbon;
optionally from 15% to 24% oxygen, from 0.1% to 5% nitrogen, and from 70% to 80% carbon;
optionally from 20% to 24% oxygen, from 0.1% to 1% nitrogen, and from 70% to 79% carbon.

The treated contact surface is treated as having a slight depth in the substrate, as XPS composition is measured through a small zone of depths. For the present purpose, the pertinent XPS analysis for the contact surface measures its composition at a depth of 5.8 Angstroms, or about 0.6 nm. Optionally in any embodiment, the interior portion and the contact surface consist essentially of the same polymer and the XPS atomic composition of the contact surface comprises from 0.1 to 30 atomic %, optionally from 2 to 30 atomic %, optionally from 5 to 20 atomic %, optionally from 10 to 20 atomic %, optionally from 13 to 16 atomic % more oxygen than the XPS atomic composition of the interior portion.

Optionally in any embodiment, the contact surface composition is measured at a depth of 0 to 20 nm, optionally 0.1 to 10 nm, optionally 0.2 to 1 nm, optionally 0.2 to 0.7 nm, optionally about 0.6 nm; and optionally the contact surface is hydrophilic and has higher cell adhesion than an untreated otherwise identical surface or biological coating coated surface. Optionally, the interior portion comprises essentially carbon and hydrogen atoms, as when it is made of a hydrocarbon. As another option, the interior portion can comprise substantial proportions of carbon, hydrogen, and a heteroatom such as oxygen, nitrogen, sulfur, chlorine, or others.

Optionally in any embodiment, the contact surface has a thickness of less than 0.6 nm and the XPS atomic composition of the interior portion 103 of the treated polymeric substrate 101 at a depth of 0.6 nm comprises from 1% to 10% oxygen.

Optionally in any embodiment, the contact surface has a thickness of less than 1.2 nm and the XPS atomic composition of the interior portion 103 of the treated polymeric substrate 101 at a depth of 1.2 nm comprises from 0.5% to 5% oxygen.

Optionally in any embodiment, the contact surface has a thickness of less than 1.7 nm and the XPS atomic composition of the interior portion 103 of the treated polymeric substrate 101 at a depth of 1.7 nm comprises from 0.3% to 3% oxygen.

Optionally in any embodiment, the contact surface has a thickness of less than 2.3 nm and the XPS atomic composition of the interior portion 103 of the treated polymeric substrate 101 at a depth of 2.3 nm comprises from 0.1% to 1% oxygen.

Optionally in any embodiment, the contact surface has a thickness of less than 2.9 nm and the XPS atomic composition of the interior portion 103 of the treated polymeric substrate 101 at a depth of 2.9 nm comprises from 0.1% to 1% oxygen.

Optionally in any embodiment, the XPS atomic composition of the contact surface comprises from 0.1 to 20 atomic %, optionally from 5 to 15 atomic %, optionally from 9 to 12 atomic %, carbon atoms to which oxygen is grafted.

Optionally in any embodiment, the contact surface comprises from 0.1 to 20 atomic %, optionally from 5 to 15 atomic %, optionally from 9 to 12 atomic %, hydrogen bond acceptor groups.

Optionally in any embodiment, the grafted oxygen is present in the form of moieties selected from C—O, CO₃, C═O, or O—C═O, optionally selected from or any combination of two or more of these, as measured by x-ray photoelectron spectroscopy (XPS).

### Contact Angle

The treatment described in this invention increases the hydrophilicity of the contact surface. Expressing the same thing another way, the treatment lowers the contact angle of the contact surface with water. (Unless otherwise indicated, the contact angles referred to in this specification are all relative to deionized water.) Higher hydrophilicity is believed to be beneficial for cell adhesion and cell growth.

Optionally in any embodiment, the surface contact angle of the contact surface is from 38° to 62°, optionally from 50° to 70°, optionally from 55° to 65°, optionally from 60° to 64°, optionally from 30° to 50°, optionally from 30 to 40°, optionally from 35° to 45°, optionally from 37° to 41°.

### Substrate

Optionally in any embodiment, the treated polymeric substrate 101 comprises a vessel 105 having a wall 106 having an inner surface 107 enclosing a lumen 108, an outer surface 109, and an interior portion 103 between and spaced from the inner surface 107 and the outer surface 109. Unless otherwise indicated in this specification, locations within the interior portion 103 are identified by their distance from the inner surface 107. The inner surface 107 optionally is generally cylindrical, and optionally the treated contact surface 102 comprises at least a portion of the inner surface 107 of the vessel 105.

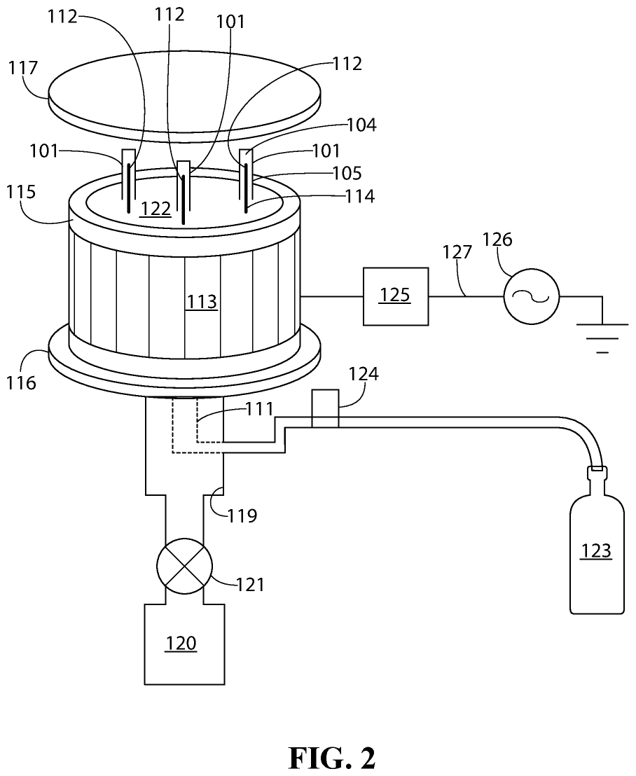

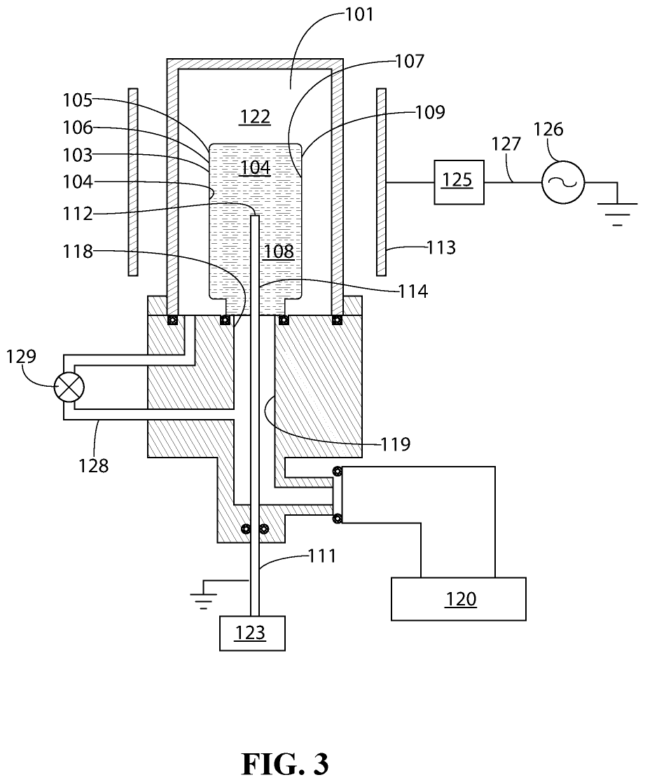

Optionally in any embodiment, the vessel 105 comprises a roller bottle as illustrated in FIGS. 1, 2, and others. Optionally, the roller bottle comprises an inner surface 107 defining the treated contact surface 102, the contact surface 102 having multiple ribs 110. Ribs or other structural complexity in part or all of the contact surface 102, for example in the cell-contacting side or end walls of the roller bottle or other vessel 105, have been found useful for increasing the surface area of the contact surface 102. Optionally in any embodiment, the vessel 105 has a volumetric capacity from 1 mL to 100 L, optionally from 100 mL to 5 L, optionally about 1 L, optionally about 2 L. Optionally in any embodiment, the treated polymeric substrate 101 can comprise a plate, a dish, a flask, a bottle as in FIGS. 1 and 3, a tube as in FIGS. 2, or any other type of lab ware or production equipment.

Optionally in any embodiment, the treated polymeric substrate 101 comprises injection moldable thermoplastic or thermosetting material, for example a thermoplastic material, for example a thermoplastic resin, for example an injection-molded thermoplastic resin. Optionally in any embodiment, the thermoplastic material comprises a hydrocarbon polymer, for example an olefin polymer, polypropylene (PP), polyethylene (PE), cyclic olefin copolymer (COC), cyclic olefin polymer (COP), polymethylpentene, polystyrene, hydrogenated polystyrene, polycyclohexylethylene (PCHE), or combinations of two or more of these, or a heteroatom-substituted hydrocarbon polymer, for example a polyester, polyethylene terephthalate (PET), polyethylene naphthalate, polybutylene terephthalate (PBT, polyvinylidene chloride (PVdC), polyvinyl chloride (PVC), polycarbonate, polylactic acid, epoxy resin, nylon, polyurethane polyacrylonitrile, polyacrylonitrile (PAN), an ionomeric resin, or any combination, composite, blend, or laminate of any two or more of the above materials. Optionally in any embodiment, the thermoplastic resin comprises polystyrene, which is commonly used for many lab ware applications, including roller bottles, microplates, petri dishes, and others.

Optionally in any embodiment, the treated polymeric substrate 101 comprises polystyrene.

### Treatment Process

Optionally, the contact surface is treated with a process comprising contacting the contact surface with a process gas and introducing radio frequency microwave, or other electrical power in the process gas adjacent to the initial contact surface to generate plasma adjacent to the initial contact surface, thereby forming a treated polymeric substrate having a treated contact surface. Optionally, the process is carried out under conditions effective to improve cell recovery of a chicken embryo cell culture from the treated contact surface, relative to the initial contact surface

In any embodiment, the treatment comprises the steps of (a) providing a substrate, for example a vessel, having a contact surface; (b) drawing a vacuum adjacent to the contact surface; (c) providing a gas comprising O2, optionally containing nitrogen, in the vicinity of the contact surface; and (d) generating a plasma from the gas, thus forming a treated contact surface. The formed contact surface is a high cell binding surface.

Optionally, if the substrate is a roller bottle or other vessel, in step (c), the gas is optionally introduced into the vessel through a gas inlet inserted into the vessel (as illustrated in FIG. XX. Optionally in this embodiment, RF is used to generate the plasma.

Surprisingly, it was found that RF power combined with use of a gas inlet introducing the gas mixture into a vessel affords great advantages in enhancing the results in cell growth experiments. The results are better than uncoated otherwise identical surfaces and also better than a Corning CellBIND™ treated surface. Not limited by the theory, when using RF power to treat a vessel without a gas inlet inserted into the vessel to deliver the gas mixture, less reactive functional groups may be generated on the surface, thus a less desired treatment may be obtained. Using a gas inlet inserted into the vessel to deliver the gas mixture helps generate more reactive functional groups on the surface, thus improving surface activation and surface uniformity to achieve better cell adhesion/cell growth results.

There are several advantages for using a RF power source versus a microwave source: Since RF operates at a lower power, there is less heating of the substrate/vessel. Because the focus of the present invention is a plasma surface treatment of plastic substrates, lower processing temperatures are desired to prevent melting/distortion of the substrate. The higher frequency microwave can also cause off-gassing of volatile substances like residual water, oligomers and other materials in the plastic substrate. This off-gassing can interfere with the treatment.

Referring to FIGS. 1-3, the present method can be carried out, in general, by providing a polymeric substrate 101 including an initial contact surface 102, contacting the initial contact surface 102 with a process gas 104 (shown as the gas source in FIG. 1, and as the gas in a vessel in FIGS. 1 and 3), and introducing radio frequency, microwave, or other plasma-generating electrical power in the process gas 104, forming a treated contact surface 102 that has improved cell recovery compared to an untreated contact surface 102.

Optionally in any embodiment, the polymeric substrate 101 includes, in addition to the initial contact surface 102, an interior portion 103 adjacent to the initial contact surface 102.

Optionally in any embodiment, the process gas 104 can be nitrogen gas, oxygen gas, or a heterogeneous gas that contains nitrogen atoms, oxygen atoms, or a combination of nitrogen and oxygen atoms, as well as other kinds of atoms. Non-limiting examples of suitable process gases 104 include oxygen gas, nitrogen gas, nitrous oxide gas, or a combination of any two or more of these. Optionally, the process gas 104 can include a carrier gas, for example a noble gas, for example helium, neon, argon, krypton, or xenon or a mixture of any two or more of these.

Optionally in any embodiment, the radio frequency or microwave electrical power is introduced in the process gas 104 adjacent to the initial contact surface 102 to generate plasma adjacent to the initial contact surface 102. As a result, a treated polymeric substrate 101 is formed having a treated contact surface 102.

Optionally in any embodiment, the process gas 104 comprises oxygen atoms, nitrogen atoms, or both oxygen and nitrogen atoms, and preferably comprises oxygen, nitrogen, nitrous oxide, or a combination of any two or more of these. Optionally in any embodiment, the process gas 104 is essentially free of water.

Optionally in any embodiment, the present method is carried out by contacting a contact surface 102 with a process gas 104. This can be done, for example, by conveying the process gas 104 through a gas inlet conduit 111 having an outlet 112 adjacent to the initial contact surface 102.

Optionally in any embodiment where radio-frequency energy is used, the frequency of the RF electrical power used for generating plasma can be from 1 to 50 MHz, optionally 13.56 MHz. Optionally in any embodiment, the electrical power used to excite the plasma can be from 1 to 1000 Watts, optionally from 100 to 900 Watts, optionally from 50 to 600 Watts, optionally 200 to 700 Watts, optionally 400 to 600 Watts, optionally 100 to 500 Watts, optionally from 500 to 700 Watts, optionally from 1 to 100 Watts, optionally from 1 to 30 Watts, optionally from 1 to 10 Watts, optionally from 1 to 5 Watts.

Optionally in any embodiment where radio frequency power is used, the radio frequency electrical power can be introduced at least in part by an external applicator 113 generally surrounding the initial contact surface 102. Optionally in any embodiment, the radio frequency electrical power is introduced at least in part by an internal applicator 114 located at least partially within the lumen 108. Optionally in any embodiment, the internal applicator 114 located at least partially within the lumen 108 further comprises a gas inlet conduit 111 for contacting the initial contact surface 102 with the process gas 104.

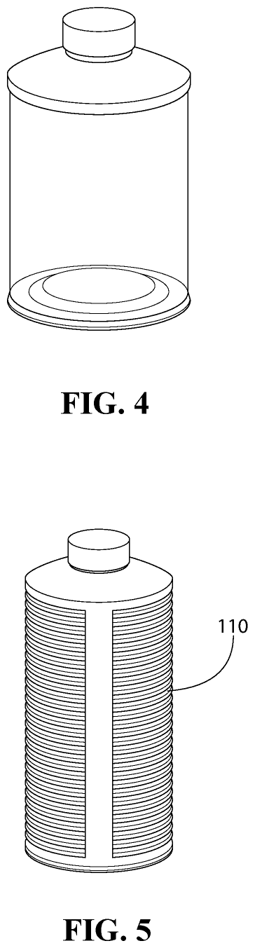

Optionally in any embodiment, apparatus as illustrated in FIG. 1 can be used to treat the initial contact surface 102 of a vessel 105. FIGS. 1 and 3 show an example of the vessel 105, configured as a roller bottle. A better view of a typical 1-liter or 2-liter capacity roller bottle is shown in FIGS. 4-6.

References in this specification to the capacity of a roller bottle or other vessel do not necessarily indicate the amount of fluid required to fill it completely full. The designated capacity of such vessels commonly allows for a headspace when the vessel is filled to its capacity. In a roller bottle, for example, the bottle is laid on its side and rolled by a mechanism when cells are being grown in the vessel so cells adhered to the contact surface 102 alternately pass through the headspace and the liquid content of the bottle, such as a growth medium, facilitating growth.

The roller bottle or other vessel 105 has a wall 106 having an inner surface 107, enclosing a lumen 108, and an outer surface 109. The vessel wall 106 has an interior portion 103 between and spaced from the inner surface 107 and the outer surface 109. At least a portion, and optionally all, of the inner surface 107 defines a contact surface 102, which is either referred to as an initial contact surface before the present treatment or a treated contact surface after the present treatment. The contact surface 102 is any part of the inner surface 107 treated according to the present disclosure.

The apparatus shown in FIG. 1, 2, or 3 is suitable for treating the vessel 105 according to any embodiment, although other apparatus can be used. This apparatus can include a cylindrical ceramic chamber 115 shown in FIGS. 1 and 2, with an aluminum bottom 116 and an aluminum lid 117 (which is closed during use, but shown open in FIG. x, as it can be when loading or unloading). The chamber 115 can be approximately 12 inches (30 cm) in diameter and 8 inches (20 cm) deep, although any other suitable dimensions can instead be used.

The pumping port 118 of the chamber 115 feeding the vacuum conduit 119 to the vacuum pump 120, optionally controlled by a valve 121, can be at the aluminum bottom 116 and can be approximately 4 inches (10 cm) in diameter, with the ½-inch (12 mm) diameter gas inlet conduit 111 concentrically protruding through the pumping port 118 into the processing area 122. A plasma screen (not shown) can be installed in over the pumping port 118 and can be constructed from copper screen and steel wool. Process gas 104 can be fed to the gas inlet conduit 111 via a gas system 123 under the chamber 115. Mass flow controllers such as 124 can be used for the compressed process gas 104.

The ceramic chamber 115 can have a copper external applicator 113 that can be concentrically wrapped around the outside of the chamber 115 and can be approximately 7 inches (18 cm) tall. The external applicator 113 can be connected to a COMDEL® matching network 125 that can allow the 50-ohm output of the COMDEL® 1000-watt RF (13.56 MHz) power supply 126 to be matched for optimal power coupling (low reflected power). COMDEL® equipment is sold by Comdel, Inc., Gloucester, Mass., USA. The power supply 126 can be attached to the COMDEL® matching network 125 via a coaxial cable 127. Two capacitance manometers (0-1 Torr and 0-100 Torr) (not shown) can be attached to the vacuum conduit 119 (also referred to as a pump line) to measure the process pressures.

The apparatus shown in FIG. 2 for treating the vessel 105 can be the same as that of FIG. 1, but as illustrated has more than one gas inlet conduit 111 to accommodate more than one vessel 105 in a single treatment cycle.

The apparatus shown in FIG. 1 or 2 optionally includes a vacuum bypass line 128 as shown in FIG. 3.

Optionally in any embodiment, lab ware configured as a flask, a bottle, or a tube can be processed in apparatus like that of FIGS. 1-3.

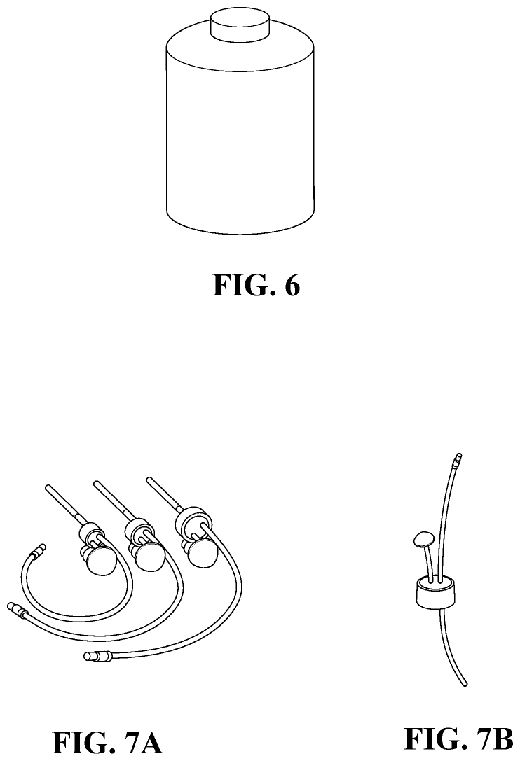

Optionally in any embodiment, lab ware configured as a plate, a microplate, a dish, or other object having relatively flat exterior surfaces to be treated can be treated in apparatus like that of FIGS. 1-3, but adapted to process flatter pieces. Optionally in any embodiment, the interior of the ceramic chamber 115 as illustrated here can be adapted as shown in FIG. 6 of WO 2016/176561 to support multiple microplates or other relatively flat objects during treatment as described in this specification. Optionally in any embodiment, the microplates or other flat objects can be oriented so the surface to be treated faces the center of the ceramic chamber 115, facilitating the application of plasma energized gas directly to the surfaces presented for treatment.

In any embodiment, the plasma is a direct plasma. Not limited to the hypothesis, direct plasma is more efficient than remote plasma in roughening the surface due to the direct interaction of the ion and the surface.

### VS Corning

Corning® CellBIND® roller bottler (described in U.S. Pat. No. 6,617,152) generated a different contact surface from the current invention. Particularly the contact surface of the present development can have a high density of small grain-like structures which are absent from the treated contact surface of the Corning® CellBIND® roller bottler. This difference can be quantified by at least one of the following roughness parameters and as described in Example 5.

Optionally, after the treatment, the contact surface has a higher Surface Area Difference A1 value, relative to the Surface Area Difference A2 value of the interior surface of the 2 L Corning® CellBIND® roller bottler. Optionally A1 is greater than 0.055%. Optionally A1>2×A2, optionally A1>5×A2, optionally A1>10×A2, optionally A1>15×A2, optionally A1>20×A2, optionally A1>25×A2, optionally A1>30×A2.

Optionally, after the treatment according to the current invention, the contact surface has a surface area difference A, greater than 0.055%, optionally from 0.06% to 2%, optionally from 0.1% to 1.5%, optionally from 0.5% to 1.2%, optionally from 0.9% to 1.1%; wherein A is determined by measurement of a 5 μm×5 μm image area on the contact surface.

Optionally, after the treatment, the Sdq1 of the contact surface is larger than the Sdq2 of the contact surface of a Cellbind roller bottle of the same size, optionally Sdq1>2×Sdq2, optionally Sdq1>3×Sdq2, optionally Sdq1>4×Sdq2.

Optionally, after the treatment, the Sds1 of the contact surface is larger than the Sds2 of the contact surface of a Cellbind roller bottle of the same size, optionally Sds1>2×Sds2, optionally Sds1>3×Sds2, optionally Sds1>4×Sds2.

Optionally, after the treatment, the Ssc1 of the contact surface is larger than the Ssc2 of the contact surface of a Cellbind roller bottle of the same size, optionally Ssc1>2×Ssc2, optionally Ssc1>3×Ssc2, optionally Ssc1>4×Ssc2, optionally Ssc1>5×Ssc2, optionally Ssc1>6×Ssc2, optionally Ssc1>7×Ssc2.

### Cell Adhesion and Cell Growth

Optionally, the substrate of this invention is used for cell growth, and the cells are harvested or recovered after the growth process is complete.

Optionally, the substrate is treated according to one embodiment using RF plasma. During the treatment, a process gas is introduced through an inlet inserted into the substrate or adjacent to the treated surface. This treatment generate high density, small-grain like structure features which increases high surface area and high roughness quantified by at least one of high Root Mean Square Surface Slope (Sdq), Density of Summits (Sds) and Mean Summit Curvature (Sdc). Cell adhesion occurs through the interaction between cell and the contact surface, high contact surface area is beneficial to cell adhesion.

Many polymer surfaces, e.g. polystyrene surface, are hydrophobic which is unfavorable to cell adhesion. Optionally the treatment according to any embodiment, incorporates more oxygen atoms into the contact surface, which increases the hydrophilicity of the surface. Hydrophilicity and high content of oxygen on surface are properties considered to enhance cell adhesion.

It is a surprising discovery that the surface roughness, higher oxygen atomic composition and hydrophilicity of the contact surface, each of which optionally can be generated by the plasma treatment described in this specification, can combine to effectively increase the cell adhesion, cell growth and cell recovery. The surface roughness is characterized by high density of small, grain-like structures, quantified by at least one of the roughness parameters of Surface Area Difference (A), Root Mean Square Surface Slope (Sdq), Density of Summits (Sds) and Mean Summit Curvature (Ssc).

The recovery rate optionally is higher than for a biological coating treated, otherwise identical substrate. The recovery rate optionally is higher than for a Corning CellBIND® substrate.

Optionally, if the substrate is embodied as a vessel, the vessel further comprises a closure. The closure can be of any kind. For example, the closure can be any stopper, cap, lid, top, cork or any combination of them. For example, a plastic or elastomer stopper can be inserted into a cap to form a closure.

Cell growth requires an aseptic environment. Frequent opening and closing the cap of the cell culture/growth vessel is one of the sources of contamination. Optionally cell culture/growth vessels (e.g. roller bottles) can be closed with an aseptic transfer cap to prevent the contamination due to opening and closing the cap during media feeding, inoculation, sample addition/collection, transferring, etc. Optionally, the closure is suitable for an aseptic process, optionally at high temperature, low temperature, autoclaving, irradiation or any other unusual conditions. For example, the closure can be an aseptic transfer cap with other accessories to eliminate the need to open the cap during the cell culture/growth process. Optionally, the closure can be a corning® aseptic transfer cap. Optionally, the closure can be a Sartorius MYCAP® closure. The MYCAP® closure comprises a silicone elastomer dispensed into a cap. The cap is assembled by inserting a tubing and a gas exchange cartridge into preformed holes located on the cap.

Optionally in any embodiment, the viability of a chicken embryo cell culture grown in contact with the treated contact surface 102 and harvested, relative to the initial contact surface 102, is at least 88%, optionally from 88% to 99%, optionally from 88% to 97%, optionally from 94% to 96%. The cell culture testing is performed according to Cell Culture, Cell Harvest and Recovery Protocol and Example 1.

Optionally in any embodiment, the recovery of a chicken embryo cell culture grown in contact with the treated contact surface 102 and harvested, relative to the initial contact surface 102, can be at least 132%, optionally from 132% to 300%, optionally from 140% to 250%, optionally from 140% to 230%. The cell culture testing is performed according to Cell Culture, Cell Harvest and Recovery Protocol and Example 1.

The process optionally improves cell recovery of a chicken embryo cell culture from the treated contact surface 102, relative to the initial contact surface 102, resulting in cell recovery from the treated contact surface 102 of at least 140% of the cells provided to the treated contact surface 102 at the beginning of the cell recovery test. The cell culture testing is performed according to Cell Culture, Cell Harvest and Recovery Protocol and Example 1.

The cells can also grow on microcarrier surfaces, another type of substrate that also increases the contact surface area. A microcarrier is a support matrix allowing for adhesive cell growth. Microcarriers are usually 125-250 micrometer spheres (beads) and their density allows them to be maintained in suspension in the medium with gentle stirring. Microcarriers or beads can be made from a number of different materials including DEAE-dextran, glass, polystyrene plastic, acrylamide, collagen, and alginate. These microcarrier or bead materials, along with different surface chemistries, can influence cellular behavior, including morphology and proliferation. There are many advantages by using microbarriers (or beads) technologies, e.g. less culture medium and less lab ware needed.

While it is important to enhance cell adhesion and cell growth, it is commonly also important to harvest the cells and retain the quality of the cells after the completion of the growth process. Optionally, when microcarriers are used, cell harvesting can be considered to involve two steps: firstly, the cells are detached from microcarriers to produce a cell-microcarrier suspension; and secondly, a further separation step leaving the cells in suspension without the microcarriers present.

Typically, the first step, i.e. cell detachment from microcarriers is accomplished by enzymatic digestion. Different enzymes can be used based on the types of microcarriers, types of cells, etc. The enzymes can be, for example, trypsin, accutase, collagenase or a trypsin-accutase mixture. During the second step, filters or centrifuges are used to separate the cells from the microcarriers.

The present invention also optionally relates to, plasma coating or treatment of the microcarrier (e.g. bead) surface to provide high hydrophilic surface to enhance cell adhesion and cell growth. The coating or treatment does not have negative impact on the cell integrity during the cell adhesion, cell growth and cell recovery process.

### Cell Culture, Cell Harvest and Recovery Protocol

The following materials, equipment, and methods are contemplated for use with the present disclosure. Materials: CELLTREAT® 1,000 mL Roller Bottle (Product 229582), CELLTREAT® T-182 Flasks (Product 229351), Medium DMEM (Gibco; Ref#1995-065), Calf serum (Gibco; Ref@ 16170-078), 1× PBS (Gibco; Ref#14190-136), 1× Trypsin with 0.18 mM EDTA diluted with 1× PBS (Gibco; Ref#25200-056), Counting slides (Bio-Rad; Cat#145-0011), Cell counter (Bio-Rad; Model TC10), Trypan Blue Solution 0.4% (Armesco, Code:K940-100ML), Penicillin Streptomycin Soln, 100× (Corning; Ref#30-002CI), competitor 2,000 mL Roller Bottle.

The selected cells were counted and split into T-182 Flasks (3/33) ×15 when received on Friday. On Monday, the 15× T-182 Flasks of cells were pooled. 10 mL of cells were added to the 1 L roller bottles and 20 mL of cells were added to the 2 L roller bottles. Roller bottles were rotated at 0.25 rpm in a humidified chamber at 39° C. with 5% CO2 in air. After 48 hours, the cells were harvested.

The harvesting of cells was performed in the following manner for 1 L roller bottles. The medium was decanted. The cells were rinsed with 25 mL of 1× PBS. Then 10 mL 1× Trypsin with 0.18 mM EDTA was added and incubated for 10 minutes. Finally 40 mL of complete medium was added. A 1 mL sample was collected and a cell count was performed.

For 2 L roller bottles, harvesting cells was performed as follows. The medium was decanted. The cells was rinsed with 50 mL of 1× PBS. Then 20 mL 1× Trypsin with 0.18 mM EDTA was added and incubated for 10 minutes. 80 mL of complete medium was added. 1 mL sample was collected and a cell count was performed.

Each sample was diluted 10× to help separate the cells. The cell samples were once again diluted 10× but in addition with 0.4% Trypan Blue to a 1:1 ratio. The 10 μL of the cell/trypan blue sample was loaded into a counting slide, which was loaded into the Bio-Rad Cell Counter and recorded.

Analysis performed compared Viable Cell Recovery, which is calculated as follows:

% Viable Cell Recovery=Total Viable Cells Harvested/Initial Total Viable Cells

Materials List

| Material | Manufacturer/Cat# |
|---|---|
| Medium DMEM | Gibco; Ref# 11995-065 |
| Calf serum | Gibco; Ref# 16170-078 |
| 1x PBS | Gibco; Ref# 14190-136 |

1x Trypsin w/0.18 mM EDTA diluted with 1xPBS from: Gibso; Ref# 25200-056

| | |
|---|---|
| Counting slides | Bio-Rad; Cat# 145-0011 |
| Cell counter | Bio-Rad; Model TC10 |
| Trypan Blue solution 0.4% | Amresco; Code: K940-100 ML |
| Penicillin Streptomycin Soln, 100X | Corning; Ref# 30-002-C1 |
| T-182 flask | Celltreat; Code: 229351 |

Experiment Design
Order cells so that they arrive on Friday. Once cells
are received, count and split to 1-182 (3/33) x15.
Monday pool 15 t-182 flasks and add 10 ml of
cells/1 L roller bottles and 20 ml/2 L roller bottles.
Roller bottles are rotated at 0.25 rpm in a humidified
chamber at 39° C. with 5% CO2 in air. Wednesday
the cells are harvested (total 48 hours).
In order to harvest:

| | 1 L Roller bottles | 2 L Roller bottles |
|---|---|---|
| 1 | Decant medium | Decant medium |
| 2 | Rinse with 25 ml 1x PBS | Rinse with 50 ml 1x PBS |
| 3 | Add 10 ml 1x Trypsin | Add 20 ml 1x Trypsin |
| | with 0.18 mM EDTA | w/0.18 mM EDTA |
| 4 | Incubate for 10 minutes | Incubate for 10 minutes |
| 5 | Add 40 ml of Complete medium | Add 80 ml of Complete medium |
| 6 | Collect 1 ml sample and count | Collect 1 ml sample and count |

Each sample is mixed 10x to help separate the cells. The cell samples are once again mixed 10x but this time with 0.4% Trypan Blue to a 1:1 ratio. 10 μl of the cell/trypan blue sample is then loaded into accounting slide and loaded into the BioRad Cell Counter and recorded.

### EXAMPLE 1

This experiment was carried out to examine the cell recovery (i.e. cell growth) improvement and contact angles due to the present surface treatment applied to a 1 L CellTreat roller bottle made of polystyrene. This experiment also compared the treatment of the current invention with competitive treatments, such as the Corning Tissue Culture Treated (TCT) roller bottle and Corning CellBIND® roller bottle, regarding cell growth. The cell line for the test was chicken embryo cells. The treatment process is described in the specification. Roller bottles 1-4 were treated according to the current invention and the parameters used are shown in Table 1a. The treated bottles were then loaded with cells as shown in Table 1b The results in Tables 2-4 show that the treatment of roller bottle 2, sometimes referred to in this specification as treatment 2, consistently afforded the best cell growth results (expressed in cell recovery data). The surface analysis shown in the following examples was performed on the roller bottles treated with the method of treatment 2, unless specified otherwise.

Water contact angles were also determined, as reported in Table 5.

TABLE 1a
Treatment Parameters

| Roller Bottle | Nitrogen | Oxygen | Power (W) | Time (s) |
|---|---|---|---|---|
| 1 | 10 | 20 | 475 | 60 |
| 2 | 10 | 10 | 600 | 60 |
| 3 | 0 | 20 | 400 | 60 |
| 4 | 0 | 10 | 500 | 90 |

TABLE 1b
Starting Cell Loading

| | Starting Viable | Starting Cells | Starting Viable |
|---|---|---|---|
| Cell Loading | (×10⁵) | (×10⁵) | (%) |
| 1^{st} round | 96.0 | 105.0 | 92.0 |
| 2nd round | 24.9 | 35.4 | 70.0 |
| 3rd round | 95.0 | 104.0 | 91.0 |

TABLE 2
Cell Recovery Results (1^{st} Round)

| Roller Bottle | Viability % | Recovery % |
|---|---|---|
| 1 | 90% | 143% |
| 2 | 81% | 228% |
| Corning TCT | 82% | 130% |
| (2 Liter) | | |
| Corning CellBIND ® | 87% | 113% |
| (2 Liter) | | |

TABLE 3
Cell Recovery Results (2^{nd} Round)

| Roller Bottle | Viability % | Recovery % |
|---|---|---|
| 1 | 96% | 162% |
| 2 | 79% | 190% |
| Corning TCT | 93% | 149% |
| (2 Liter) | | |
| Corning CellBIND ® | — | — |
| (2 Liter) | | |

TABLE 4
Cell Recovery Results (3^{rd} Round)

| Roller Bottle | Viability % | Recovery % |
|---|---|---|
| 2 | 91% | 81% |
| 3 | 76% | 76% |
| 4 | 82% | 63% |
| Corning TCT | 91% | 64% |
| (2 Liter) | | |
| Corning CellBIND ® | 94% | 63% |
| (2 Liter) | | |

TABLE 5
Water Contact Angles

| Roller Bottle | Surface Contact Angle |
|---|---|
| 1 | 61° |
| 2 | 52° |
| 3 | 39° |
| 4 | 38° |
| Corning TC (2 Liter) | 62° |
| Corning CellBIND ® (2 Liter) | 39° |

### EXAMPLE 2

### XPS Surface Analysis of Treated Roller Bottle of the Invention and Untreated Roller Bottle

This example was carried out to determine the chemical composition and chemical bonding of the contact surface of an untreated 1 L CELLTREAT™ roller bottle made of polystyrene and the contact surface of a treated otherwise identical roller bottle B. The surface treating process for the roller bottle of FIG. 6 is the same as treatment 2 in Example 1. The XPS was performed on one area (the middle area) of the contact surface of bottle A and four areas of the contact surface of bottle B. The four areas are shown in FIG. 6. The concentration of the elements was determined from high resolution spectra. These XPS results are summarized in Table 6.

TABLE 6
Atomic Concentrations (in atomic %)

| Sample | C | N | O | Si |
|---|---|---|---|---|
| Bottle A (middle) | 90.1 | 0.1 | 7.4 | 2.4 |
| Bottle B (top) | 71.1 | 0.6 | 22.8 | 5.4 |
| Bottle B (middle) | 72.4 | 0.7 | 21.9 | 5.0 |
| Bottle B (bottom) | 73.6 | 0.6 | 21.2 | 4.6 |
| Bottle B (base) | 71.2 | 0.6 | 22.7 | 5.5 |

The results show that the treatment of the current invention results in three times more oxygen on the treated surface than on an identical untreated surface.

The chemical bonding information is shown in Table 7.

TABLE 7
Carbon and Silicon Chemical States (in atomic %)

| | Carbon | | | | | | | |
|---|---|---|---|---|---|---|---|---|
| | | | | | | aromatic | Silicon | |
| Sample | C—(C, H) | C—O | C = O | O—C = O | CO₃ | loss | silicone | silicate |
| Bottle A (middle) | 80.0 | 4.9 | 1.0 | — | — | 4.1 | 1.9 | 0.6 |
| Bottle B (top) | 59.3 | 6.8 | 2.2 | 0.7 | 1.2 | 1.0 | 3.2 | 2.1 |
| Bottle B (middle) | 61.7 | 7.0 | 2.0 | 0.4 | 0.8 | 0.5 | 3.0 | 2.0 |
| Bottle B (bottom) | 61.8 | 7.2 | 2.4 | 0.8 | 0.8 | 0.6 | 2.8 | 1.8 |
| Bottle B (base) | 59.4 | 6.7 | 2.3 | 1.0 | 0.9 | 1.0 | 3.2 | 2.3 |

The chemical states of the detected elements were determined from the high resolution spectra. For the elements C and Si, the spectra were curve fit to estimate the relative amounts of each element in different oxidation states. The curve fit results are shown on the individual spectra and summarized in Table 7.

### EXAMPLE 3

### XPS In-Depth Analysis of Treated Roller Bottle of the Invention and Untreated Roller Bottle

This example was used to determine the in-depth chemical composition of the contact surface of the treated roller bottle B of this invention.

Survey spectra were acquired for the contact surface of the treated roller bottle B. A depth profile was acquired using 1 kV Ar+. The results are shown in Table 8. This beam voltage was selected to minimize preferential sputtering of oxygen atoms. While this minimizes preferential sputtering, it does not completely remove this artifact. Consequently, the oxygen concentrations during the depth profiles are expected to be higher than the measured values. Note that the depth scales in this study assumed that the samples sputtered at the same rate as a 825 Å spin cast thin film of polystyrene.

TABLE 8
Atomic Concentration

| Depth (Å) | C | N | O | Si |
|---|---|---|---|---|
| 0.0 | 72.5 | 0.4 | 22.4 | 4.7 |
| 5.8 | 94.0 | — | 3.9 | 2.1 |
| 11.5 | 97.5 | — | 1.4 | 1.1 |
| 17.3 | 98.5 | — | 0.8 | 0.7 |
| 23.0 | 98.7 | — | 0.6 | 0.7 |
| 28.8 | 98.9 | — | 0.6 | 0.5 |
| 34.6 | 99.3 | — | 0.3 | 0.4 |
| 40.3 | 99.4 | — | 0.3 | 0.3 |
| 46.1 | 99.3 | — | 0.3 | 0.4 |
| 51.8 | 99.5 | — | 0.3 | 0.2 |
| 57.6 | 99.5 | — | 0.3 | 0.2 |
| 63.4 | 99.6 | — | 0.3 | 0.2 |
| 69.1 | 99.5 | — | 0.4 | 0.2 |
| 74.9 | 99.5 | — | 0.4 | 0.1 |
| 80.6 | 99.5 | — | 0.4 | — |
| 86.4 | 99.5 | — | 0.5 | — |
| 95.0 | 99.7 | — | 0.3 | — |
| 104 | 99.6 | — | 0.4 | — |
| 112 | 99.7 | — | 0.3 | — |
| 121 | 99.6 | — | 0.4 | — |
| 130 | 99.7 | — | 0.3 | — |
| 143 | 99.6 | — | 0.4 | — |
| 156 | 99.8 | — | 0.2 | — |
| 168 | 99.5 | — | 0.5 | — |
| 181 | 99.7 | — | 0.3 | — |
| 194 | 99.8 | — | 0.2 | — |
| 214 | 99.8 | — | 0.2 | — |

^{a} Normalized to 100% of the elements detected. XPS does not detect H or He.
^{b} A dash line “—” indicates the element is not detected.

### EXAMPLE 4

### Atomic Force Microscope (AFM) Analysis of Treated Roller Bottle of the Invention and Untreated Roller Bottle

This example was to compare the roughness of the interior surface of a treated roller bottle of the current invention versus the roughness of the interior surface of an untreated otherwise identical roller bottle.

The roughness was evaluated using Atomic Force Microscope (AFM). AFM images were collected using a Dimension Icon AFM instrument (Bruker, Santa Barbara, Calif., USA). The instrument is calibrated against a NIST traceable standard.

The samples were identified as A for the treated roller bottle and B for the untreated roller bottle. The samples were prepared at locations approximately halfway up the sides of each bottle by cutting with a razor. One 20 μm×20 μm area was imaged on the inside surface. Top views of these areas are shown along with the roughness measurements in FIGS. 8 and 9. The topography differences of these images are shown in gray scale image, where low features are shown in a darker shade and higher features are shown in lighter shades or white. The z ranges are noted on the vertical scale bar on the right side of the images. Perspective (3-D) views of these surfaces are also included with vertical exaggerations noted in the captions (FIGS. 10 and 11). One 2 μm×0.5 μm area on each sample was also imaged with higher lateral resolution (FIGS. 12-15). The instrument conditions are shown in Table 9.

TABLE 9
Instrument Conditions

| | |
|---|---|
| Instrument | Dimension Icon SPM (Bruker) |
| Analysis Mode | Soft Tapping Mode |
| AFM Probe | EBD_NCH_13 (Nanotools) |
| Data Post-Processing | 2^{nd} order flattening |

The roughness analyses were performed and expressed as height, spatial, and hybrid parameters. The results are summarized in Table 10 and Table 11.

TABLE 10
Roughness Results - Height Parameters

| | | | | | Surface |
|---|---|---|---|---|---|
| | | R_{q} | Rₐ | Rₘₐₓ | Area Diff |
| Sample ID | Area | (nm) | (nm) | (nm) | (%) |
| A | 20 μm × 20 μm | 3.54 | 2.72 | 99.0 | 1.15 |
| B | | 2.85 | 1.77 | 119 | 0.044 |
| A | 2 μm × 0.5 μm | 2.14 | 1.74 | 18.3 | 12.2 |
| B | | 0.94 | 0.75 | 7.63 | 0.145 |

TABLE 11
Roughness Results - Spatial and Hybrid Parameters

| | | Sₐₗ | S_{dq} | S_{ds} | S_{sc} |
|---|---|---|---|---|---|
| Sample ID | Area | (nm) | (°) | (/μm²) | (/μm) |
| A | 20 μm × 20 μm | 196 | 8.68 | 11.3 | 7.47 |
| B | | 1230 | 1.77 | 1.18 | 0.895 |
| A | 2 μm × 0.5 μm | 14.1 | 27.4 | 1817 | 114 |
| B | | 129 | 3.08 | 751 | 13.8 |

The results in the Tables 10-11 and the images of FIGS. 8-9 clearly show the textural differences between the two surfaces. The interior surface of the treated bottle of the current invention exhibit high density of small, grain-like structures that were absent on the untreated bottle surface. This can also be quantified by the roughness parameters, the treated roller bottle of the current invention has higher Sdq value which indicates narrower spaced textural components, consistent with the high density of small, grain-like structures. It is believed that the small, grain-like structures increase the contact surface area more efficiently than fewer larger, wider spaced bump structures. Particularly the surface area difference of the treated surface A1 is greatly larger than the surface difference area of the untreated surface A2. Other roughness parameters such as Rq, Ra, Rmax, Sdq, Sds and Ssc are also increased after treatment.

This trend was more pronounced on a smaller scale (i.e. 2 μm×0.5 μm). Textural differences between the two surfaces were also more apparent in the smaller sized images; a high density of small grain-like structures was observed on the treated bottle and were absent on the untreated bottle (FIGS. 12 to 15).

Not limited by the theory, it is believed that the increased contact surface area, quantified by the larger surface area difference value and other higher roughness parameters after the treatment described in this example above, is at least one of the reasons for enhanced cell adhesion; and higher cell adhesion is considered to enhance higher cell growth on the surface.

The estimated uncertainties of the roughness values provided are within ±5-10% (at an approximate level of confidence of 95% using a coverage factor of k=2). Roughness data below 2 nm should be viewed as “semi-quantitative” unless a separate z-height calibration in this range is performed. “Semi-quantitative” data still allows for comparisons between samples as the precision of the measurement is about ±10%. (The uncertainty of the absolute roughness values however is not determined.) Please note that the uncertainty estimates provided assume that there is no variability in roughness between different locations sampled.

The following descriptions of the parameters related to roughness are applied to the whole disclosure.

The Bruker Dimension Icon AFM/SPM acquires and stores 3-dimensional representations of surfaces in a digital format. These surfaces can be analyzed in a variety of ways.

The Nanoscope software can perform a roughness analysis of any AFM or SPM image. The product of this analysis is a single color page reproducing the selected image in top view. To the upper right of the image is the “Image Statistics” box, which lists the calculated characteristics of the whole image minus any areas excluded by a stopband (a box with an X through it). Similar additional statistics can be calculated for a selected portion of the image and these are listed in the “Box Statistics” in the lower right portion of the page. What follows is a description and explanation of these statistics.

### General Height Parameters

Z Range (Rₚ): The difference between the highest and lowest points in the image. This value is not corrected for tilt in the plane of the image; therefore, plane fitting or flattening the data will change the value.

Mean: The average of all of the Z values in the imaged area. This value is not corrected for tilt in the plane of the image; therefore, planefitting or flattening the data will change this value.

RMS (Rq): This is the standard deviation of the Z values (or RMS roughness) in the image. It is calculated according to the formula:

Rq=√{Σ(Zi−Zavg)2/N}

where Zavg is the average Z value within the image; Zi is the current value of Z; and N is the number of points in the image. This value is not corrected for tilt in the plane of the image; therefore, plane fitting or flattening the data will change this value.

Mean roughness (Ra): This is the mean value of the surface relative to the Center Plane and is calculated using the formula:

Ra=[1/(LxLy)]∫0Ly∫0Lx{f(x,y)}dxdy

where f(x,y) is the surface relative to the Center Plane, and Lx and Ly are the dimensions of the surface.

Max height (Rmax): This is the difference in height between the highest and lowest points of the surface relative to the Mean Plane.

Surface area: This is the area of the 3-dimensional surface of the imaged area. It is calculated by taking the sum of the areas of the triangles formed by 3 adjacent data points throughout the image.

Surface area diff: This is the amount that the Surface area is in excess of the imaged area. It is expressed as a percentage and is calculated according to the formula:

Surface area diff=100[(Surface area/S12)−1]

where S1 is the length (and width) of the scanned area minus any areas excluded by stopbands.

Center Plane: A flat plane that is parallel to the Mean Plane. The volumes enclosed by the image surface above and below the center plane are equal.

Mean Plane: The image data has a minimum variance about this flat plane. It results from a first order least squares fit on the Z data.

### Spatial Parameters

Fastest Decay Autocorrelation Function (Sal): This optional spatial parameter is defined as the length of the fastest decay of the 20% of the autocorrelation function, in any direction. A high value for Sal indicates the surface is dominated by low frequency components

Texture Direction of Surface (Std): This optional spatial parameter is the angle of the dominant lay of the surface, relative to the Y axis. This parameter is determined from the Angular Power Spectral Density Function.

Texture Aspect Ratio (Str): This optional spatial parameter is defined as the ratio of the fastest decay to the slowest decay to correlation 20% of the autocorrelation function. Str will be closer to 0 for surfaces with a strong lay; Str will be closer to 1 for surfaces having a uniform texture.

### Hybrid Parameters

Root Mean Square Surface Slope (Sdq): is a measure of the slopes that make up the surface texture, evaluated over all directions. It includes amplitude and spacing components. Lower Sdq values may indicate wider spaced textural components:

$$= \sqrt{[ 1 / {\int }_{0} Ly {\int }_{0} Lx \{ {( \partial / \partial ( ) )}^{2} + {( \partial / \partial ( ) )}^{2} \} ]}$$

Density of Summits, (Sds): the number of summits per unit area. Summits are derived from peaks. A peak is defined as any point, above all 8 nearest neighbors. Summits are constrained to be separated by at least 1% of the minimum “X” or “Y” dimension comprising the 3D measurement area. Additionally, summits are only found above a threshold that is 5% of Sz above the mean plane.

Mean Summit Curvature, (Ssc): the mean summit curvature for the various peak structures. Ssc is evaluated for each summit and then averaged over the area:

Ssc=1/N∫∫[{(∂2z(x,y))/∂x2}+{(∂2z(x,y))/∂y2}dxdy]

### EXAMPLE 5

### Atomic Force Microscope (AFM) Analysis of Treated Roller Bottle of the Invention and Untreated Roller Bottle

This example was to compare the roughness of the interior surface (i.e. contact surface) of a treated roller bottle of the current invention versus the roughness of the interior surface (i.e. contact surface) of a same size CellBIND® roller bottle.

The roughness was evaluated using Atomic Force Microscope (AFM). AFM images were collected using a Dimension Icon AFM instrument (Bruker, Santa Barbara, Calif., USA). The instrument is calibrated against a NIST traceable standard.

The samples were identified as A for the treated roller bottle of the current invention and B for Corning CellBIND® roller bottle. The samples were prepared at locations approximately halfway up the sides of each bottle by cutting with a razor. One 5 μm×5 μm area was imaged on the inside surfaces. Top views of these areas are shown along with the roughness measurements in FIGS. 16 and 17. The z ranges are noted on the vertical scale bar on the right side of the images. Perspective (3-D) views of these surfaces are also included with vertical exaggerations noted in the captions (FIGS. 18 and 19). The instrument conditions are shown in Table 9.

The roughness analyses were performed and expressed as height, spatial, and hybrid parameters. The results are summarized in Table 12 and Table 13.

TABLE 12
Roughness Results - Height Parameters

| | | | | | Surface |
|---|---|---|---|---|---|
| | | R_{q} | Rₐ | Rₘₐₓ | Area Diff |
| Sample ID | Area | (nm) | (nm) | (nm) | (%) |
| A | 5 μm × 5 μm | 1.84 | 1.45 | 27.3 | 1.07 |
| B | | 2.86 | 2.26 | 28.1 | 0.055 |

TABLE 13
Roughness Results - Spatial and Hybrid Parameters

| | | Sₐₗ | S_{dq} | S_{ds} | S_{sc} |
|---|---|---|---|---|---|
| Sample ID | Area | (nm) | (°) | (/□m²) | (/□m) |
| A | 5 μm × 5 μm | 372 | 8.39 | 134 | 22.9 |
| B | | 402 | 1.90 | 44.4 | 3.62 |

The results and the images clearly show the textural differences between the two surfaces. The interior surface of the treated roller bottle of the current invention exhibit a high density of small, grain-like structures which were largely absent on the Corning CellBIND® surface (FIGS. 16 and 17). This can also be quantified by the roughness parameters. Particularly the surface area difference of the treated surface A1 of the current invention is greatly larger than the surface difference area A2 of the Corning CellBIND®. Other roughness parameters such as Sdq, Sds and Ssc are also increased after treatment. Not limited by the theory, it is believed that the increased contact surface area, quantified by larger surface area difference value, is at least one of the reasons for improved cell adhesion and higher cell adhesion is positively correlated to higher cell growth on the surface. It is consistent with the experimental observation that the treated contact surface of the current invention results in higher cell recovery than Corning CellBIND® roller bottle described in Example 1.

The estimated uncertainties of the roughness values provided are within ±5-10% (at an approximate level of confidence of 95% using a coverage factor of k=2). Roughness data below 2 nm should be viewed as “semi-quantitative” unless a separate z-height calibration in this range is performed. “Semi-quantitative” data still allows for comparisons between samples as the precision of the measurement is about ±10%. (The uncertainty of the absolute roughness values however is not determined.) Please note that the uncertainty estimates provided assume that there is no variability in roughness between different locations sampled.

While the technology has been described in detail and with reference to specific examples and embodiments thereof, it will be apparent to one skilled in the art that various changes and modifications can be made therein without departing from the spirit and scope thereof. Additional disclosure is provided in the claims, which are considered to be a part of the present description, each claim defining an optional and optional embodiment.

1.-29. (canceled)

30. A polymeric substrate comprising a vessel having a wall having an inner surface enclosing a lumen, an outer surface, and an interior portion between the inner surface and the outer surface, and
a treated contact surface, wherein the contact surface comprises at least a portion of the inner surface of the vessel, in which the contact surface has a roughness quantified by at least one of the four parameters below:
a surface area difference A, greater than 0.055%, optionally from 0.06% to 2%, optionally from 0.1% to 1.5%, optionally from 0.5% to 1.2%, optionally from 0.9% to 1.1%;
a root mean square surface slope, Sdq, greater than 1.9°, optionally from 2° to 20°, optionally from 4° to 15°, optionally from 6° to 12°, optionally from 7° to 10°, optionally from 7° to 9°;
a density of summits, Sds, greater than 44.4/μm², optionally from 45/μm² to 200/μm², optionally from 50/μm² to 180/μm², optionally from 60/μm² to 170/μm², optionally from 70/μm² to 160/μm², optionally from 80/μm² to 160/μm², optionally from 90/μm² to 150/μm², optionally from 100/μm² to 150/μm², optionally from 110/μm² to 150/μm², optionally from 120/μm² to 150/μm²; or
a mean summit curvature, Ssc, greater than 3.62/μm, optionally from 4/μm to 50/μm, optionally from 6/μm to 45/μm, optionally from 8/μm to 40/μm, optionally from 10/μm to 35/μm², optionally from 12/μm² to 30/μm, optionally from 14/μm to 30/μm, optionally from 16/μm to 30/μm, optionally from 18/μm to 30/μm, optionally from 20x/μm to 25/μm;
wherein the parameters A, Sdq, Sds, and Ssc are each determined by measurement of a 5 μm×5 μm image area on the contact surface; and
in which the XPS atomic composition of the interior portion of the polymeric substrate comprises a smaller proportion of oxygen and a greater proportion of carbon than the XPS atomic composition of the contact surface, wherein the XPS atomic composition of the contact surface is determined to a depth of 5.8 Angstroms.

31. The substrate of claim 30, in which the interior portion and the contact surface consist essentially of the same polymer and;
the XPS atomic composition of the contact surface comprises from 0.1 to 30 atomic %, optionally from 2 to 30 atomic %, optionally from 5 to 20 atomic %, optionally from 10 to 20 atomic %, optionally from 13 to 16 atomic % more oxygen than the XPS atomic composition of the interior portion;
the XPS atomic composition of the contact surface comprises from 0.1 to 20 atomic %, optionally from 5 to 15 atomic %, optionally from 9 to 12 atomic %, carbon atoms to which oxygen is grafted;
the contact surface comprises from 0.1 to 20 atomic %, optionally from 5 to 15 atomic %, optionally from 9 to 12 atomic %, hydrogen bond acceptor groups; and
the grafted oxygen is present in the form of moieties selected from C—O, CO₃, C═O, or O—C═O, or any combination of two or more of these, as measured by x-ray photoelectron spectroscopy (XPS).

32. The substrate of claim 30, in which the contact surface has a thickness of 0 to 20 nm, optionally 0.1 to 10 nm, optionally 0.2 to 1 nm, optionally 0.2 to 0.7 nm, optionally about 0.6 nm; and in which the contact surface is hydrophilic and has higher cell adhesion than an untreated otherwise identical surface or biological coating coated surface; and the interior portion consists essentially of carbon and hydrogen atoms.

33. The substrate of claim 32, in which the contact surface has a thickness of less than 0.6 nm and the XPS atomic composition of the interior portion at a depth of 0.6 nm comprises from 1% to 10% oxygen; alternatively
in which the contact surface has a thickness of less than 1.2 nm and the XPS atomic composition of the interior portion of the treated polymeric substrate at a depth of 1.2 nm comprises from 0.5% to 5% oxygen; alternatively
in which the contact surface has a thickness of less than 1.7 nm and the XPS atomic composition of the interior portion of the treated polymeric substrate at a depth of 1.7 nm comprises from 0.3% to 3% oxygen; alternatively
in which the contact surface has a thickness of less than 2.3 nm and the XPS atomic composition of the interior portion of the treated polymeric substrate at a depth of 2.3 nm comprises from 0.1% to 1% oxygen; alternatively
in which the contact surface has a thickness of less than 2.3 nm and the XPS atomic composition of the interior portion of the treated polymeric substrate at a depth of 2.3 nm comprises from 0.1% to 1% oxygen; alternatively in which the XPS atomic composition of the interior portion of the treated polymeric substrate at a depth of 2.9 nm comprises from 0.1% to 1% oxygen.

34. The substrate of claim 30, in which the surface contact angle of the contact surface is from 38° to 62°, optionally from 50° to 70°, optionally from 55° to 65°, optionally from 60° to 64°, optionally from 30° to 50°, optionally from 30 to 40°, optionally from 35° to 45°, optionally from 37° to 41°.

35. The substrate of claim 30, in which the vessel comprises a flask, a bottle, or a tube.

36. The substrate of claim 35, in which the vessel further comprises a closure, optionally a stopper, cap, lid, top, cork or any combination of them.

37. The substrate of claim 36, in which the closure is formed by inserting a plastic or elastomer stopper into a cap.

38. The substrate of claim 35, in which the inner surface is generally cylindrical.

39. The substrate of claim 38, in which the vessel comprises a roller bottle.

40. The substrate of claim 39, in which the inner surface defining the treated contact surface has multiple ribs.

41. The substrate of claim 30, in which the vessel has a volumetric capacity from 1 mL to 100 L, optionally from 100 mL to 5 L, optionally 1 L, optionally 2 L.

42. The substrate of claim 30, in which the treated polymeric substrate comprises thermoplastic material, for example a thermoplastic resin, for example an injection-molded thermoplastic resin; and in which the thermoplastic resin optionally comprises polystyrene.

43. The substrate of claim 42, in which the thermoplastic material comprises a hydrocarbon polymer, for example an olefin polymer, polypropylene (PP), polyethylene (PE), cyclic olefin copolymer (COC), cyclic olefin polymer (COP), polymethylpentene, polystyrene, hydrogenated polystyrene, polycyclohexylethylene (PCHE), or combinations of two or more of these, or a heteroatom-substituted hydrocarbon polymer, for example a polyester, polyethylene terephthalate (PET), polyethylene naphthalate, polybutylene terephthalate (PBT), polyvinylidene chloride (PVdC), polyvinyl chloride (PVC), polycarbonate, polylactic acid, epoxy resin, nylon, polyurethane polyacrylonitrile, polyacrylonitrile (PAN), an ionomeric resin, or any combination, composite, blend, or laminate of any two or more of the above materials.

44. The substrate of claim 30, in which the contact surface is treated under conditions effective to improve cell recovery of a chicken embryo cell culture from the treated contact surface relative to the initial contact surface;
the treatment comprising contacting the contact surface with a process gas; and introducing radio frequency electrical power in the process gas adjacent to the initial contact surface to generate plasma adjacent to the initial contact surface, thereby forming a treated polymeric substrate having a treated contact surface; and
wherein cell recovery of a chicken embryo cell culture is determined by contacting a cell culture with Medium DMEM containing calf serum, placing 10 mL of the cell culture and culture medium in a 1 L roller bottle or 20 mL of the cell culture and culture medium in a 2 L roller bottle, rolling the bottle at 0.25 rpm in a humidified chamber at 39° C. with 5% CO2 in air for 48 hours, harvesting the cells using Trypsin with 0.18 mM EDTA, and determining recovery by mixing the sample with 0.4% Trypan Blue and loading it into BioRad Cell Counter and recording.

45. The substrate of claim 44, the process gas comprises oxygen atoms, nitrogen atoms, or both oxygen and nitrogen atoms, and preferably comprises oxygen, nitrogen, nitrous oxide, or a combination of any two or more of these and wherein the process gas is essentially free of water.

46. The substrate of claim 45, in which the contact surface is contacted with a process gas by conveying the process gas through a gas inlet conduit having an outlet adjacent to the initial contact surface.

47. The substrate of claim 44, in which the Sdq1 of the contact surface is larger than the Sdq2 of an untreated otherwise identical contact surface, optionally Sdq1>2×Sdq2, optionally Sdq1>3×Sdq2, optionally Sdq1>4×Sdq2, optionally Sdq1>5×Sdq2, optionally Sdq1>6×Sdq2, optionally Sdq1>7×Sdq2, optionally Sdq1>8×Sdq2, optionally Sdq1>9×Sdq2, optionally Sdq1>10×Sdq2, optionally Sdq1>11×Sdq2;
in which the Sds1 of the contact surface is larger than the Sds2 of an untreated otherwise identical contact surface, optionally Sds1>2×Sds2, optionally Sds1>3×Sds2, optionally Sds1>4×Sds2, optionally Sds1>5×Sds2, optionally Sds1>6×Sds2, optionally Sds1>7×Sds2, optionally Sds1>8×Sds2, optionally Sds1>9×Sds2, optionally Sds1>10×Sds2, optionally Sds1>11×Sds2, optionally Sds1>12×Sds2;
in which the Ssc1 of the contact surface is larger than the Ssc2 of an untreated otherwise identical contact surface, optionally Ssc1>2×Ssc2, optionally Ssc1>3×Ssc2, optionally Ssc1>4×Ssc2, optionally Ssc1>5×Ssc2, optionally Ssc1>6×Ssc2, optionally Ssc1>7×Ssc2, optionally Ssc1>8×Ssc2, optionally Ssc1>9×Ssc2, optionally Ssc1>10×Ssc2, optionally Ssc1>11×Ssc2, optionally Ssc1>12×Ssc2;
in which the Rq1 of the contact surface is larger than the Rq2 of an untreated otherwise identical contact surface;
in which the Ra1 of the contact surface is larger than the Ra2 of an untreated otherwise identical contact surface; in which the Surface Area Difference A1 of the contact surface is larger than the Surface Area Difference A2 of an untreated otherwise identical contact surface, optionally A1>2×A2, optionally A1>5×A2, optionally A1>10×A2, optionally A1>15×A2, optionally A1>20×A2, optionally A1>25×A2, optionally A1>30×A2, optionally A1>2×A2, optionally A1>35A2, optionally A1>40×A2, optionally A1>50×A2, optionally A1>60×A2, optionally A1>70×A2, optionally A1>80×A2, optionally A1>90×A2, optionally A1>100×A2, optionally A1>110×A2;
or any combination thereof.

48. The substrate of claim 47, in which the Surface Area Difference A1 of the contact surface is larger than the Surface Area Difference A2 of the contact surface of a Cellbind roller bottle of the same size, optionally A1>2×A2, optionally A1>5×A2, optionally A1>10×A2, optionally A1>15×A2, optionally A1>20×A2, optionally A1>25×A2, optionally A1>30×A2;
in which the Sdq1 of the contact surface is larger than the Sdq2 of the contact surface of a Cellbind roller bottle of the same size, optionally Sdq1>2×Sdq2, optionally Sdq1>3×Sdq2, optionally Sdq1>4×Sdq2;
in which the Sds1 of the contact surface is larger than the Sds2 of the contact surface of a Cellbind roller bottle of the same size, optionally Sds1>2×Sds2, optionally Sds1>3×Sds2, optionally Sds1>4×Sds2;
in which the Ssc1 of the contact surface is larger than the Ssc2 of the contact surface of a Cellbind roller bottle of the same size, optionally Ssc1>2×Ssc2, optionally Ssc1>3×Ssc2, optionally Ssc1>4×Ssc2, optionally Ssc1>5×Ssc2, optionally Ssc1>6×Ssc2, optionally Ssc1>7×Ssc2;
or any combination thereof.

49. The substrate of claim 44, in which the viability of a chicken embryo cell culture grown in contact with the treated contact surface and harvested is, relative to the initial contact surface, at least 88%, optionally from 88% to 99%, optionally from 88% to 97%, optionally from 94% to 96%;
the recovery of a chicken embryo cell culture grown in contact with the treated contact surface and harvested is, relative to the initial contact surface, at least 132%, optionally from 132% to 300%, optionally from 140% to 250%, optionally from 140% to 230%;
or both;
wherein cell recovery of a chicken embryo cell culture is determined by contacting a cell culture with Medium DMEM containing calf serum, placing 10 mL of the cell culture and culture medium in a 1 L roller bottle or 20 mL of the cell culture and culture medium in a 2 L roller bottle, rolling the bottle at 0.25 rpm in a humidified chamber at 39° C. with 5% CO2 in air for 48 hours, harvesting the cells using Trypsin with 0.18 mM EDTA, and determining recovery by mixing the sample with 0.4% Trypan Blue and loading it into BioRad Cell Counter and recording.
